(19) United States
(12) Patent Application Publication
(10) Pub. No.: US 2016/0158306 A1
(43) Pub. Date: Jun. 9, 2016

(54) HERBAL COMPOSITION FOR INCREASING FERTILITY IN MEN AND A METHOD OF SYNTHESIZING THE SAME

(71) Applicant: Fariba Sepahvand, Khoramabad (IR)

(72) Inventor: Fariba Sepahvand, Khoramabad (IR)

(21) Appl. No.: 15/044,678

(22) Filed: Feb. 16, 2016

(51) Int. Cl. A61K 36/9068; A61K 36/71; A61K 9/00

(52) U.S. Cl. CPC A61K 36/9068; A61K 9/0095; A61K 36/71

(57) ABSTRACT

The embodiments of the present invention provide herbal syrup comprising Nigella sativa (Nigella) seed extract and Zingiber officinale (ginger) root extract. The Nigella seeds and ginger roots are identified in a herbarium and confirmed. The Nigella seeds and ginger roots are cleaned and disinfected. After disinfection the Nigella seeds and ginger roots are grinded separately to obtain powder. The powders of Nigella seed and ginger root are treated in a percolator machine to extract thymoquinone and gingerol. The Nigella seed extract and ginger extract are mixed and herbal syrup is made for the patients with abnormal sperm parameters. The dosage of the herbal syrup is 7 ml. The syrup is administered daily during breakfast for 4 months. The sperm count increases gradually from 15 million/ml, 43 million/ml, 89 million/ml and 120 million/ml after four month of administration.

### BACKGROUND

1. Technical Field

The embodiments herein are generally related to a regenerative drug or medicinal composition. The embodiments herein are particularly related to an alternative remedy/herbal based composition for increasing natural fertility parameters/factors in human beings. The embodiments herein are more particularly related to a herbal composition for targeting abnormal sperm count in infertile men.

2. Description of the Related Art

Approximately 15% of couples attempting their first pregnancy do not succeed. Most medical authorities define these individual as infertile if the couples are unable to achieve a pregnancy even after having a year of unprotected sexual relationship. Conception is normally achieved within 12 months in 80-85% couples who do not use contraceptive measures and persons not able to conceive after this time are generally regarded as possibly infertile. The infertile individuals must be examined medically. Data reveals that 30% of infertility cases are found in males alone. Therefore the failure of conception due the defects in fertility of infertility in males is at least partly responsible in about 50% of infertile couples.

Sperm cells are produced by a repeated cell division in the coiled tubules within the testicles at a rate of appropriately 100 million per day. The sperm cell production is a lengthy process. The sperm cell production takes about 3 months. The sperm cell production begins with a division of the sperm cell and ends with an appearance of mature sperm in the semen. The sperm cells takes about 2 to 20 days in passing through the epididymis, during which time the sperm cells are matured and become capable of swimming and fertilizing eggs. The volume of liquid from the testes and epididymis is less than 5% of the total semen volume. The seminal vesicle contributes about 65% of the semen volume and the prostate gland contributes about 25% of the semen volume. The average semen volume for healthy men ejaculating every two days is 3 ml and the sperm concentration is 85 million per ml. During ejaculation, the sperm and the prostrate fluid comes out first and the seminal vesicle fluid follows afterwards. The seminal vesicle fluid coagulates to provide the semen with a lumpy gel like appearance. After 10 minutes or later, a liquefaction occurs and the gel disappears.

Male infertility may be caused by many conditions that affect the production of functional sperm. The most common cause is varicocele (i.e. hardening of the veins that drain the testes) which accounts for about 40% of cases and is treated surgically. Testicular failure accounts for approximately 10% of cases and results from numerous causes including malignancy, mumps, Kleinfelters syndrome, injury and radio/chemo therapy. Hyperspermia or increased seminal fluid volume, also accounts for about 10% of cases. Endocrine diseases affecting spermatogenesis account for approximately 9% of cases and usually involve pituitary or adrenal hypoplasia or hyperthyroidism. Obstruction of the ejaculatory duct accounts for about 5% of cases and sperm auto-antibodies for 1 to 2%.

Approximately 13% of men have untreatable sterility, 11% have treatable conditions and 76% have disorders of sperm production or function which do not usually have clearly defined effective treatments. Most of the persons have no sperm in their semen (azoospermia) among the 13% of men with untreatable sterility. Azoospermia condition arises when the tubules which produce sperm in the testes are not developed or have been irreversibly damaged. Inability to produce sperm or azoospermia is associated with a failure of the testes to descend into the scrotum during childhood, inflammation of the testes or treatment with certain drugs. In some sterile men, sperm are produced in normal numbers but they are either not motile (do not swim) or lack cellular structures necessary for the penetration and fertilization of eggs. The non-motile sperm and sperm cells lacking cellular structures are detected by microscope examination of the shape of the sperm and reported as abnormal morphology. Some men with failure of sperm production do not produce normal amounts of the male sex hormone, called testosterone. The general health and sexual performance is improved by a treatment with testosterone. Over three quarters of men investigated for fertility have sperm present in the semen but in lower numbers than normal-oligospermia (38%) or in adequate numbers but with reduced motility (33%).

Oligospermia is a condition associated with an abnormally low number of sperm in the ejaculate of the male. The normal range of sperm count is between 20 million/ml and 200 million/ml. The sperm count below 20 million/ml in an individual indicates oligospermia. Common causes of oligospermia are stress, smoking of tobacco (nicotine damages sperms), lead (workers in printing press have low sperm count), hot climates, saunas, hot bath, wearing of tight underwear and other situations in which scrotal temperature may be raised, varicocele and consumption of alcohol.

One treatment method is intracytoplasmic sperm injection (ICSI). ICSI is the most successful for of treatment for infertile men. ICSI is used in nearly half of all people undergoing in vitro fertilization treatment. ICSI have several disadvantages. ICSI is a more expensive procedure than in vitro fertilization (IVF). ICSI has been in use for a shorter time than IVF. So medical experts are still engaged in learning and identifying the possible effects of ICSI. The same risks associated with standard IVF procedure, such as multiple births and ectopic pregnancy are observed in ICSI. There is a higher risk of congenital conditions such as cerebral palsy in the baby. The risk rises from 3% for naturally conceived children to about six percent after IVF or IC SI. One study has revealed that a higher rate of abnormalities in ICSI. According to a study report, a higher rate of abnormalities is found in ICSI babies when compared with that of IVF babies. During natural conception, only the hardest sperm manages to travel great distances and break through the membrane of an egg to fertilize it. Weaker sperm do not fertilize the egg. As the ICSI bypass this natural selection process, there is an increased risk of rare genetic problems carried by the sperm being passed onto the child. The ICSI procedure is more expensive procedure than IVF.

In vitro fertilization or fertilization is a process by which an egg is fertilized by sperm outside the body in vitro (in glass). A disadvantage with IVF is that success is not guaranteed. Indeed, less than 50% of all couples with an IVF cycle achieves a pregnancy. Another disadvantage of IVF is that there are associated risks of infection, anesthetic risks and hemorrhage because the process involves an operative procedure. The ovarian hyperstimulation syndrome (OHSS) is also a risk that is only associated with treatment where the ovaries are stimulated through the use of medications to male multiple eggs. Hence along with male patient the female partner should undergo medical procedures and potential side effects. Further a mild reaction to fertility drugs may involve hot flushes, feeling irritable, headache and restlessness.

Herbal compositions have been used in aiding fertility since ancient times. Ancient records indicate the use of herbal composition for the treatment of infertility and miscarriage.

No individual herb is considered useful especially for promoting fertility. The design of the herbal formula has been varied over the centuries, based on prevailing theories and available resources, individual practitioners and there are preferences for particular herbs. These aforementioned factors account for some of the variations among formulas that are recommended. The differences among the individuals being treated accounts for the greatest variation in the selection of herbs and formulas to be used.

Ginger (Zingiber officinale) is a flowering plant, in the family Zingiberaceae whose rhizome ginger root or ginger is widely used as a spice or a herbal medicine. It is an herbaceous perennial which grows annual stems about a meter tall bearing narrow green leaves and yellow flowers. Ginger is indigenous to South China and was spread eventually to the Asia and Africa. The characteristics odor and the flavor of ginger is caused by a mixture of zingerone, shogaols and gingerols. Volatile oils that compose one to three percent of the weight of fresh ginger. Ginger belongs to Zingiberaceae family. Ginger is a native to South East Asia. Ginger is used as an additive and seasoning in foods for more than 4000 years and also has been used for medical purposes over 2005 years. It has been become a widely used material in Europe since 13th century. Although Ginger is considered that the root of Ginger is comestible. The usable part of Ginger is the inflated underground stem called Rhizome.

Ginger is used as dietary supplement, food additive and flavoring agent worldwide. Although Food and Drug Administration (FDA) has considered ginger as a safe supplemented, primary studies on animals in laboratory shows that Ginger inhibits platelet aggregation and it is effective against blood clotting. So Ginger consumption is prescribed for those who take anti-coagulation medicines such as warfarin.

Nigella sativa is often called black cumin, is an annual flowering plant in the family Ranunculaceae, a native to South and Southwest Asia. Nigella sativa grows to 20-30 cm tall with finely divided, linear (leaves). The flowers are delicate and usually colored pale blue and white with five to ten petals. The black caraway fruit is a large and inflated capsule composed of three to seven united follicles, each containing numerous seeds which are used as spice. Nigella sativa oil contains conjugated linoleic acid (18:2), thymoquinone, nigellone (di-thymoquinone), melanthin, nigilline and trans-anethole.

Nigella sativa ethanol extracts increases follicle graph and chemiluminescence which lead to female fertility. The essence of Nigella sativa contains a wide range of medical properties which are anti-microbial, anti-tumor, anti-virus, anti-inflammatory, anti-oxidants, muscle relaxation and expansion. Nigella seeds extract has anti-worm, bactericidal, anti-fungal, menstruation organizer, purgative, milk secretion enhancing, anti-inflammatory and analgesic properties.

Prior arts disclose that the main composites/active ingredients in the Nigella sativa seeds are dihydroergotamine thymoquinone, thymoquinone, and hydroquinone. Further the prior arts disclose that Nigella oil has following effects on non-pregnant rats and rabbits. The Nigella oil is menstruation enhancer and uterus stimulant.

The common herbs like Zingiber officinale (Ginger) and Nigella sativa (Nigella) are easily available. Also the herbal extracts from the Zingiber officinale (Ginger) and Nigella sativa (Nigella) have no side effects unlike the medical treatment procedure, chemical compositions or pharmaceuticals compositions.

Hence, there is a need for an herbal composition for enhancing/increasing the male fertility, specifically increasing the sperm count in infertile men. Also there is a need for an herbal composition for enhancing/increasing the male fertility by increasing the sperm count without any side effects. Further there is a need for a method of synthesizing the herbal composition for enhancing/increasing the male fertility.

The above mentioned shortcomings, disadvantages and problems are addressed herein and which will be understood by reading and studying the following specification.

### OBJECTIVES OF THE EMBODIMENTS

The primary objective of the embodiment herein is to synthesize a herbal composition comprising Nigella sativa (Nigella) seed extract and Zingiber officinale (Ginger) ginger extract for enhancing/increasing the sperm count in infertile human male patients.

Another objective of the embodiment herein is to provide a herbal composition comprising Nigella sativa (Nigella) seed extract and Zingiber officinale (Ginger) ginger extract for enhancing/increasing the sperm count without any side effects.

Yet another objective of the embodiment herein is to provide the herbal composition comprising Nigella sativa (Nigella) seed extract and Zingiber officinale (Ginger) ginger extract in the form of syrup.

Yet another objective of the embodiment herein is to provide the herbal composition comprising Nigella sativa (Nigella) seed extract and Zingiber officinale (Ginger) ginger extract which is inexpensive and remarkably cost effective as compared to the existing medication and compositions.

Yet another objective of the embodiment herein is to provide the herbal composition comprising Nigella sativa (Nigella) seed extract and Zingiber officinale (Ginger) ginger extract which is easily synthesized.

Yet another objective of the embodiment herein is to analyze the effect of Nigella sativa extract and Zingiber officinale extract on the patients with abnormal sperm parameters.

These and other objects and advantages of the present invention will become readily apparent from the following detailed description taken in conjunction with the accompanying drawings.

### SUMMARY

The various embodiments herein provide a herbal composition for enhancing/increasing the male fertility, specifically increasing the sperm count in infertile men. The herbal composition for enhancing/increasing the male fertility by increases the sperm count without any side effects.

According to one embodiment herein, the method of synthesizing a herbal syrup composition, for targeting abnormal sperm parameters in infertile male patients, comprises of the following steps. The Nigella sativa seeds and Zingiber officinale roots are collected. The Nigella sativa seeds and the Zingiber officinale roots are cleaned and disinfected.

The Nigella sativa seeds and Zingiber officinale roots are grinded separately to obtain Nigella sativa seed powder and Zingiber officinale root powder. The thymoquinone is extracted from Nigella sativa seed powder using an alcohol solvent in a percolator machine. The percolator machine Nigella sativa seed powder alcohol extract is obtained. The thymoquinone is an active ingredient of Nigella sativa seed powder alcohol extract. The gingerol is extracted from Zingiber officinale root powder using an alcohol solvent in a percolator machine. In the percolator machine Zingiber officinale root powder alcohol extract is obtained. The gingerol is an active ingredient of Zingiber officinale root powder alcohol extract. The Nigella sativa seed powder alcohol extract and the Zingiber officinale powder alcohol extract are collected separately. The Nigella sativa seed powder alcohol extract and the Zingiber officinale powder alcohol extract are mixed in a predetermined concentration to obtain a mixture of Nigella sativa seed powder alcohol extract and the Zingiber officinale powder alcohol extract. A plurality of additives are added to the mixture of Nigella sativa seed powder alcohol extract and the Zingiber officinale powder alcohol extract to obtain the herbal syrup composition. The herbal syrup composition is administered to infertile male patients with abnormal sperm parameters. The infertile male patients with abnormal sperm parameters are subjected to sperm analysis.

According to one embodiment herein, the alcohol solvent is ethanol.

According to one embodiment herein, the herbal syrup composition is administered to infertile male patients with abnormal sperm parameters in an amount of 7 ml.

According to one embodiment herein, the herbal syrup composition is administered to infertile male patients with abnormal sperm parameters during morning meal. The morning meal is breakfast.

According to one embodiment herein, the herbal syrup composition is administered to infertile male patients with abnormal sperm parameters for a period of 4 months.

According to one embodiment herein, the herbal syrup composition comprises the Nigella sativa seed powder alcohol extract and Zingiber officinale root powder alcohol extract in a concentration of 1-10% v/v.

According to one embodiment herein, the herbal syrup composition comprises the plurality of additives in a concentration of 150-200 ml.

According to one embodiment herein, the sperm analysis of the male patients after one month of administering the herbal syrup composition illustrates a sperm count in a semen sample of male patient in a concentration of 15.78 million/ml. The motile sperms in the male patient semen sample are present in a concentration of 52.14%. The progressive sperm in the male patient semen sample are present in a concentration of 47.14%.

According to one embodiment herein, the sperm analysis of the male patients after second month of administering the herbal syrup composition illustrates a sperm count in a semen sample of male patient in a concentration of 43.19 million/ml. The motile sperms in the male patient semen sample are present in a concentration of 65.79%. The progressive sperm in the male patient semen sample are present in a concentration of 61.62%.

According to one embodiment herein, the sperm analysis of the male patients after third month of administering the herbal syrup composition illustrates a sperm count in a semen sample of male patient in a concentration of 89.52 million/ml. The motile sperms in the male patient semen sample are present in a concentration of 69.80%. The progressive sperm in the male patient semen sample are present in a concentration of 61.43%.

According to one embodiment herein, the herbal syrup composition for targeting abnormal sperm parameters in infertile male patients comprises of Nigella sativa seed powder alcohol extract, a Zingiber officinale root powder alcohol extracts and a plurality of additives. The thymoquinone is an active ingredient present in the Nigella sativa seed powder alcohol extract. The gingerol is an active ingredient present in the Zingiber officinale root powder alcohol extract. The herbal syrup composition is administered to infertile male patients with abnormal sperm parameters. The infertile male patients with abnormal sperm parameters are subjected to sperm analysis.

According to one embodiment herein, the herbal syrup composition is administered to infertile male patients with abnormal sperm parameters in an amount of 7 ml.

According to one embodiment herein, the herbal syrup composition is administered to infertile male patients with abnormal sperm parameters during morning meal. The morning meal is breakfast.

According to one embodiment herein, the herbal syrup composition is administered to infertile male patients with abnormal sperm parameters for a period of 4 months.

According to one embodiment herein, the herbal syrup composition comprises the Nigella sativa seed extract and Zingiber officinale root extract in a concentration of 1-10% v/v.

According to one embodiment herein, the herbal syrup composition comprises the plurality of additives in a concentration of 150-200 ml.

According to one embodiment herein, the sperm analysis of the male patients after one month of administering the herbal syrup composition illustrates a sperm count in a semen sample of male patient in a concentration of 15.78 million/ml. The motile sperms in the male patient semen sample are present in a concentration of 52.14%. The progressive sperm in the male patient semen sample are present in a concentration of 47.14%.

According to one embodiment herein, the sperm analysis of the male patients after second month of administering the herbal syrup composition illustrates a sperm count in a semen sample of male patient in a concentration of 43.19 million/ml. The motile sperms in the male patient semen sample are present in a concentration of 65.79%. The progressive sperm in the male patient semen sample are present in a concentration of 61.62%.

According to one embodiment herein, the sperm analysis of the male patients after third month of administering the herbal syrup composition illustrates a sperm count in a semen sample of male patient in a concentration of 89.52 million/ml. The motile sperms in the male patient semen sample are present in a concentration of 69.80%. The progressive sperm in the male patient semen sample are present in a concentration of 61.43%.

According to one embodiment herein, the effect of Nigella sativa (Nigella) seed extract and Zingiber officinale (Ginger) ginger extract is analyzed on the patients with abnormal sperm parameter.

According to one embodiment herein, the Nigella sativa (Nigella) seeds and Zingiber officinale (Ginger) root are obtained. The Nigella sativa (Nigella) seeds and Zingiber officinale (Ginger) roots are identified in a herbarium of Faculty of Pharmacy Tehran University of Medical Sciences. The herbarium confirms the Nigella sativa (Nigella) seeds and Zingiber officinale (Ginger) roots. The seeds of Nigella sativa (Nigella) and roots of Zingiber officinale (Ginger) roots are subjected to cleaning and disinfection. The disinfected seeds of Nigella sativa (Nigella) and the disinfected roots of Zingiber officinale (Ginger) roots are grinded separately to obtain a powder of Nigella sativa (Nigella) seeds and Zingiber officinale (Ginger) roots. The extract from the powder of Nigella sativa (Nigella) seeds and Zingiber officinale (Ginger) roots is extracted using alcohol solvent in a percolator machine. The alcohol solvent is ethanol. The alcohol extracts of Nigella sativa (Nigella) seeds and Zingiber officinale (Ginger) roots are obtained separately. The alcohol extract of Nigella sativa (Nigella) seeds and Zingiber officinale (Ginger) roots is mixed and added along with additives to form the herbal syrup.

According to one embodiment herein, the active ingredients in the extract of Nigella sativa (Nigella) seeds is thymoquinone. The active ingredient in the extract of Zingiber officinale (Ginger) roots is gingerol.

According to one embodiment herein, the herbal syrup comprises a mixture of 1-10% v/v Nigella sativa (Nigella) seed extract and Zingiber officinale (Ginger) root extract and 150-250 ml of syrup components and additives.

According to one embodiment herein, the effect of herbal syrup comprising Nigella sativa (Nigella) seed extract and Zingiber officinale (Ginger) root extract analyzed on the abnormal sperm parameters of infertile men. The sample of the study comprises of 100 male patients. The male patients with defective sperm parameters are referred to the hospital. The male patients are selected from Sina hospital, Iran. The male patients selected for study must not be detected with azoospermia.

According to one embodiment herein, the male patients are enrolled after obtaining consent and explaining the objectives of the study. The male patients are divided into two groups. Each group comprises of 50 people. The patients having semen sample less than normal (based on World Health Organization (WHO)) are put in two groups. In the first group the male patients are administered the herbal syrup comprising Nigella sativa (Nigella) seed extract and Zingiber officinale (Ginger) root extract. The second group or control group of the male patients is administered placebo. Both the group of male patients are not administered any pharmaceutical drugs or compositions for treating infertility or abnormal sperm parameters during the term of the study.

According to one embodiment herein, the first group the male patients are administered the herbal syrup comprising Nigella sativa (Nigella) seed extract and Zingiber officinale (Ginger) root extract for 4 months. The dosage or quantity of the herbal syrup is 7 ml. The herbal syrup is administered during breakfast daily for 4 months. After each month the number of sperm and activity test of sperm is analyzed in the male patients. After 4 months the male patients are advised to go to the infertility center or hospital to test the effect of the herbal syrup on the abnormal sperm parameter.

According to one embodiment herein, the herbal syrup composition comprising Nigella sativa seed extract and Zingiber officinale root extract in has a positive effect on the testicles of the male patients. The herbal syrup composition comprising Nigella sativa seed extract and Zingiber officinale root extract increases fertility in the male patients by increasing sperm production and motility in sperm.

According to one embodiment herein, the active ingredient of Nigella sativa seed extract is thymoquinone. The active ingredient of Zingiber officinale root extract is gingerol. The thymoquinone and gingerol has a positive effect on testis. The thymoquinone and gingerol stimulate testis of male patients to produce sperm cells and increase the motility of the sperm cells. The second group or control group of the male patients is administered placebo. The second group/control group male patients do not show any effect on the sperm production and sperm motility.

According to one embodiment herein, the semen sample test is conducted in the first group of patients administered with herbal syrup comprising Nigella sativa seed extract and Zingiber officinale root extract.

According to one embodiment herein, semen sample test is conducted after first month of administering herbal syrup comprising Nigella sativa seed extract and Zingiber officinale root extract. The semen sample test after first month of administering herbal syrup illustrates number of sperm cells are 15 million/ml of semen.

According to one embodiment herein, semen sample test is conducted after second month of administering herbal syrup comprising Nigella sativa seed extract and Zingiber officinale root extract. The semen sample test after second month of administering herbal syrup illustrates number of sperm cells are 43 million/ml of semen.

According to one embodiment herein, semen sample test is conducted after third month of administering herbal syrup comprising Nigella sativa seed extract and Zingiber officinale root extract. The semen sample test after third month of administering herbal syrup illustrates number of sperm cells are 89 million/ml of semen.

According to one embodiment herein, semen sample test is conducted after fourth month of administering herbal syrup comprising Nigella sativa seed extract and Zingiber officinale root extract. The semen sample tests after fourth month of administering herbal syrup illustrates number of sperm cells are 120 million/ml of semen.

After first month of administering herbal syrup, the male patients are analyzed. The result illustrates the effect of herbal syrup (herbal syrup comprising Nigella sativa seed extract and Zingiber officinale root extract) administered for one month on sperm count, motile sperm and progressive sperms in the male patient with abnormal sperm parameters. The result illustrates the sperm count in the semen of male patient is 15.78 million/ml of semen sample. The motile sperms in the male patient's semen sample are present in 52.14%. The progressive sperm in the male patient semen sample are present in 47.14%. The sperm count, motile sperm and progressive sperm are present below the normal levels or range recommended by WHO. The male patient has a condition of oligospermia.

After second month of administering herbal syrup, the male patients are analyzed. The result illustrates the effect of herbal syrup (herbal syrup comprising Nigella sativa seed extract and Zingiber officinale root extract) administered for two month on sperm count, motile sperm and progressive sperms in the male patient with abnormal sperm parameters. The result illustrates the sperm count in the semen of male patient is 43.19 million/ml of semen sample. The motile sperms in the male patients semen sample are present in 65.79%. The progressive sperm in the male patient semen sample are present in 61.62%. The sperm count, motile sperm and progressive sperm are present in a normal levels or range recommended by WHO. The male patient is not reported to have oligospermia.

After third month of administering herbal syrup the male patients are analyzed. The result illustrates the effect of herbal syrup (herbal syrup comprising Nigella sativa seed extract and Zingiber officinale root extract) administered for three month on sperm count, motile sperm and progressive sperms in the male patient with abnormal sperm parameters. The result illustrates the sperm count in the semen of male patient is 89.52 million/ml of semen sample. The motile sperms in the male patients semen sample are present in 69.80%. The progressive sperm in the male patient semen sample are present in 61.43%. The sperm count, motile sperm and progressive sperm are present in a normal levels or range recommended by WHO. The male patient is not reported to have oligospermia.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart explaining a method of synthesizing a herbal syrup comprising Nigella sativa seed extract and Zingiber officinale root extract, according to one embodiment herein.

FIG. 2 illustrates a computer aided semen analysis system report showing semen analysis result of male patient after first month of administering herbal syrup, according to one embodiment herein.

FIG. 3 illustrates a computer aided semen analysis system report showing semen analysis result of male patient after second month of administering herbal syrup, according to one embodiment herein.

FIG. 4 illustrates a computer aided semen analysis system report showing semen analysis result of male patient after third month of administering herbal syrup, according to one embodiment herein.

Although the specific features of the present invention are shown in some drawings and not in others. This is done for convenience only as each feature may be combined with any or all of the other features in accordance with the present invention.

### DETAILED DESCRIPTION OF THE EMBODIMENTS

In the following detailed description, reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a herbal composition for enhancing/increasing the male fertility, specifically increasing the sperm count in infertile men. The herbal composition for enhancing/increasing the male fertility by increases the sperm count without any side effects.

According to one embodiment herein, the method of synthesizing a herbal syrup composition, for targeting abnormal sperm parameters in infertile male patients, comprises of the following steps. The Nigella sativa seeds and Zingiber officinale roots are collected. The Nigella sativa seeds and the Zingiber officinale roots are cleaned and disinfected. The Nigella sativa seeds and Zingiber officinale roots are grinded separately to obtain Nigella sativa seed powder and Zingiber officinale root powder. The thymoquinone is extracted from Nigella sativa seed powder using an alcohol solvent in a percolator machine. The percolator machine Nigella sativa seed powder alcohol extract is obtained. The thymoquinone is an active ingredient of Nigella sativa seed powder alcohol extract. The gingerol is extracted from Zingiber officinale root powder using an alcohol solvent in a percolator machine. In the percolator machine Zingiber officinale root powder alcohol extract is obtained. The gingerol is an active ingredient of Zingiber officinale root powder alcohol extract. The Nigella sativa seed powder alcohol extract and the Zingiber officinale powder alcohol extract are collected separately. The Nigella sativa seed powder alcohol extract and the Zingiber officinale powder alcohol extract are mixed in a predetermined concentration to obtain a mixture of Nigella sativa seed powder alcohol extract and the Zingiber officinale powder alcohol extract. A plurality of additives are added to the mixture of Nigella sativa seed powder alcohol extract and the Zingiber officinale powder alcohol extract to obtain the herbal syrup composition. The herbal syrup composition is administered to infertile male patients with abnormal sperm parameters. The infertile male patients with abnormal sperm parameters are subjected to sperm analysis.

According to one embodiment herein, the alcohol solvent is ethanol.

According to one embodiment herein, the herbal syrup composition is administered to infertile male patients with abnormal sperm parameters in an amount of 7 ml.

According to one embodiment herein, the herbal syrup composition is administered to infertile male patients with abnormal sperm parameters during morning meal. The morning meal is breakfast.

According to one embodiment herein, the herbal syrup composition is administered to infertile male patients with abnormal sperm parameters for a period of 4 months.

According to one embodiment herein, the herbal syrup composition comprises the Nigella sativa seed powder alcohol extract and Zingiber officinale root powder alcohol extract in a concentration of 1-10% v/v.

According to one embodiment herein, the herbal syrup composition comprises the plurality of additives in a concentration of 150-200 ml.

According to one embodiment herein, the sperm analysis of the male patients after one month of administering the herbal syrup composition illustrates a sperm count in a semen sample of male patient in a concentration of 15.78 million/ml. The motile sperms in the male patient semen sample are present in a concentration of 52.14%. The progressive sperm in the male patient semen sample are present in a concentration of 47.14%.

According to one embodiment herein, the sperm analysis of the male patients after second month of administering the herbal syrup composition illustrates a sperm count in a semen sample of male patient in a concentration of 43.19 million/ml. The motile sperms in the male patient semen sample are present in a concentration of 65.79%. The progressive sperm in the male patient semen sample are present in a concentration of 61.62%.

According to one embodiment herein, the sperm analysis of the male patients after third month of administering the herbal syrup composition illustrates a sperm count in a semen sample of male patient in a concentration of 89.52 million/ml. The motile sperms in the male patient semen sample are present in a concentration of 69.80%. The progressive sperm in the male patient semen sample are present in a concentration of 61.43%.

According to one embodiment herein, the herbal syrup composition for targeting abnormal sperm parameters in infertile male patients comprises of Nigella sativa seed powder alcohol extract, a Zingiber officinale root powder alcohol extracts and a plurality of additives. The thymoquinone is an active ingredient present in the Nigella sativa seed powder alcohol extract. The gingerol is an active ingredient present in the Zingiber officinale root powder alcohol extract. The herbal syrup composition is administered to infertile male patients with abnormal sperm parameters. The infertile male patients with abnormal sperm parameters are subjected to sperm analysis.

According to one embodiment herein, the herbal syrup composition is administered to infertile male patients with abnormal sperm parameters in an amount of 7 ml.

According to one embodiment herein, the herbal syrup composition is administered to infertile male patients with abnormal sperm parameters during morning meal. The morning meal is breakfast.

According to one embodiment herein, the herbal syrup composition is administered to infertile male patients with abnormal sperm parameters for a period of 4 months.

According to one embodiment herein, the herbal syrup composition comprises the Nigella sativa seed extract and Zingiber officinale root extract in a concentration of 1-10% v/v.

According to one embodiment herein, the herbal syrup composition comprises the plurality of additives in a concentration of 150-200 ml.

According to one embodiment herein, the sperm analysis of the male patients after one month of administering the herbal syrup composition illustrates a sperm count in a semen sample of male patient in a concentration of 15.78 million/ml. The motile sperms in the male patient semen sample are present in a concentration of 52.14%. The progressive sperm in the male patient semen sample are present in a concentration of 47.14%.

According to one embodiment herein, the sperm analysis of the male patients after second month of administering the herbal syrup composition illustrates a sperm count in a semen sample of male patient in a concentration of 43.19 million/ml. The motile sperms in the male patient semen sample are present in a concentration of 65.79%. The progressive sperm in the male patient semen sample are present in a concentration of 61.62%.

According to one embodiment herein, the sperm analysis of the male patients after third month of administering the herbal syrup composition illustrates a sperm count in a semen sample of male patient in a concentration of 89.52 million/ml. The motile sperms in the male patient semen sample are present in a concentration of 69.80%. The progressive sperm in the male patient semen sample are present in a concentration of 61.43%.

According to one embodiment herein, the effect of Nigella sativa (Nigella) seed extract and Zingiber officinale (Ginger) root extract is analyzed on the patients with abnormal sperm parameter.

According to one embodiment herein, the Nigella sativa (Nigella) seeds and Zingiber officinale (Ginger) root are obtained. The Nigella sativa (Nigella) seeds and Zingiber officinale (Ginger) roots are identified in a herbarium of Faculty of Pharmacy Tehran University of Medical Sciences. The herbarium confirms the Nigella sativa (Nigella) seeds and Zingiber officinale (Ginger) roots. The seeds of Nigella sativa (Nigella) and roots of Zingiber officinale (Ginger) are subjected to cleaning and disinfection. The disinfected seeds of Nigella sativa (Nigella) and the disinfected roots of Zingiber officinale (Ginger) roots are grinded separately to obtain a powder of Nigella sativa (Nigella) seeds and Zingiber officinale (Ginger) roots. The extract from the powder of Nigella sativa (Nigella) seeds and Zingiber officinale (Ginger) roots is extracted using alcohol solvent in a percolator machine. The alcohol solvent is ethanol. The alcohol extracts of Nigella sativa (Nigella) seeds and Zingiber officinale (Ginger) roots are obtained separately. The alcohol extract of Nigella sativa (Nigella) seeds and Zingiber officinale (Ginger) roots is mixed and added along with additives to form the herbal syrup.

According to one embodiment herein, the active ingredients in the extract of Nigella sativa (Nigella) seeds is thymoquinone. The active ingredient in the extract of Zingiber officinale (Ginger) roots is gingerol.

According to one embodiment herein, the herbal syrup comprises a mixture of 1-10% v/v Nigella sativa (Nigella) seed extract and Zingiber officinale (Ginger) root extract and 150-250 ml of syrup components and additives.

According to one embodiment herein, the effect of herbal syrup comprising Nigella sativa (Nigella) seed extract and Zingiber officinale (Ginger) root extract analyzed on the abnormal sperm parameters of infertile men. The sample of the study comprises of 100 male patients. The male patients with defective sperm parameters are referred to the hospital. The male patients are selected from Sina hospital, Iran. The male patients selected for study must not be detected with azoospermia.

According to one embodiment herein, the male patients are enrolled after obtaining consent and explaining the objectives of the study. The male patients are divided into two groups. Each group comprises of 50 people. The patients having semen sample less than normal (based on World Health Organization (WHO)) are put in two groups. In the first group the male patients are administered the herbal syrup comprising Nigella sativa (Nigella) seed extract and Zingiber officinale (Ginger) root extract. The second group or control group of the male patients is administered placebo. Both the group of male patients are not administered any pharmaceutical drugs or compositions for treating infertility or abnormal sperm parameters during the term of the study.

According to one embodiment herein, the first group the male patients are administered the herbal syrup comprising Nigella sativa (Nigella) seed extract and Zingiber officinale (Ginger) root extract for 4 months. The dosage or quantity of the herbal syrup is 7 ml. The herbal syrup is administered during breakfast daily for 4 months. After each month the number of sperm and activity test of sperm is analyzed in the male patients. After 4 months the male patients are advised to go to the infertility center or hospital to test the effect of the herbal syrup on the abnormal sperm parameter.

According to one embodiment herein, the herbal syrup composition comprising Nigella sativa seed extract and Zingiber officinale root extract in has a positive effect on the testicles of the male patients. The herbal syrup composition comprising Nigella sativa seed extract and Zingiber officinale root increases fertility in the male patients by increasing sperm production and motility in sperm.

According to one embodiment herein, the active ingredient of Nigella sativa seed extract is thymoquinone. The active ingredient of Zingiber officinale root extract is gingerol. The thymoquinone and gingerol has a positive effect on testis. The thymoquinone and gingerol stimulate testis of male patients to produce sperm cells and increase the motility of the sperm cells. The second group or control group of the male patients is administered placebo. The second group/control group male patients do not show any effect on the sperm production and sperm motility.

According to one embodiment herein, the semen sample test is conducted in the first group of patients administered with herbal syrup comprising Nigella sativa seed extract and Zingiber officinale root extract.

According to one embodiment herein, semen sample test is conducted after first month of administering herbal syrup comprising Nigella sativa seed extract and Zingiber officinale root extract. The semen sample test after first month of administering herbal syrup illustrates number of sperm cells are 15 million/ml of semen.

According to one embodiment herein, semen sample test is conducted after second month of administering herbal syrup comprising Nigella sativa seed extract and Zingiber officinale root extract. The semen sample test after second month of administering herbal syrup illustrates number of sperm cells are 43 million/ml of semen.

According to one embodiment herein, semen sample test is conducted after third month of administering herbal syrup comprising Nigella sativa seed extract and Zingiber officinale root extract. The semen sample test after third month of administering herbal syrup illustrates number of sperm cells are 89 million/ml of semen.

According to one embodiment herein, semen sample test is conducted after fourth month of administering herbal syrup comprising Nigella sativa seed extract and Zingiber officinale root extract. The semen sample tests after fourth month of administering herbal syrup illustrates number of sperm cells are 120 million/ml of semen.

After first month of administering herbal syrup, the male patients are analyzed. The result illustrates the effect of herbal syrup (herbal syrup comprising Nigella sativa seed extract and Zingiber officinale root extract) administered for one month on sperm count, motile sperm and progressive sperms in the male patient with abnormal sperm parameters. The result illustrates the sperm count in the semen of male patient is 15.78 million/ml of semen sample. The motile sperms in the male patient's semen sample are present in 52.14%. The progressive sperm in the male patient semen sample are present in 47.14%. The sperm count, motile sperm and progressive sperm are present below the normal levels or range recommended by WHO. The male patient has a condition of oligospermia.

After second month of administering herbal syrup, the male patients are analyzed. The result illustrates the effect of herbal syrup (herbal syrup comprising Nigella sativa seed extract and Zingiber officinale root extract) administered for two month on sperm count, motile sperm and progressive sperms in the male patient with abnormal sperm parameters. The result illustrates the sperm count in the semen of male patient is 43.19 million/ml of semen sample. The motile sperms in the male patients semen sample are present in 65.79%. The progressive sperm in the male patient semen sample are present in 61.62%. The sperm count, motile sperm and progressive sperm are present in a normal levels or range recommended by WHO. The male patient is not reported to have oligospermia.

After third month of administering herbal syrup the male patients are analyzed. The result illustrates the effect of herbal syrup (herbal syrup comprising Nigella sativa seed extract and Zingiber officinale root extract) administered for three month on sperm count, motile sperm and progressive sperms in the male patient with abnormal sperm parameters. The result illustrates the sperm count in the semen of male patient is 89.52 million/ml of semen sample. The motile sperms in the male patients semen sample are present in 69.80%. The progressive sperm in the male patient semen sample are present in 61.43%. The sperm count, motile sperm and progressive sperm are present in a normal levels or range recommended by WHO. The male patient is not reported to have oligospermia.

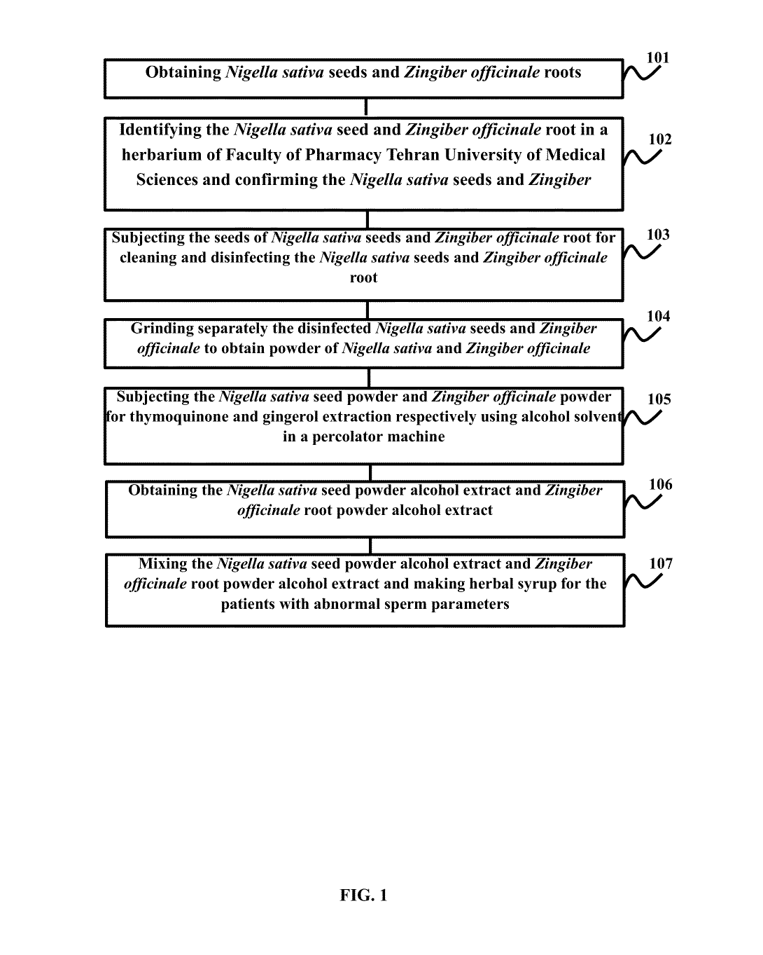

FIG. 1 illustrates a flow chart explaining a method of synthesizing a herbal syrup comprising Nigella sativa seed extract and Zingiber officinale extract, according to one embodiment herein. The Nigella sativa seeds and Zingiber officinale roots are obtained (101). The Nigella sativa seeds and Zingiber officinale roots are identified in a herbarium of Faculty of Pharmacy Tehran University of medical Sciences and the Nigella sativa seeds and Zingiber officinale roots are confirmed (102). The Nigella sativa seeds and Zingiber officinale roots are subjected for cleaning and disinfection (103). The Nigella sativa seeds and Zingiber officinale roots are grinded separately to obtain a powder (104). The powder of Nigella sativa seeds and powder of Zingiber officinale roots is subjected to thymoquinone and gingerol extraction using alcohol solvent in a percolator machine (105). The Nigella sativa seed powder alcohol extract and Zingiber officinale root powder alcohol extract is obtained (106). The Nigella sativa seed powder alcohol extract and Zingiber officinale root powder alcohol extract are mixed and herbal syrup is made for the patients with abnormal sperm parameters (107).

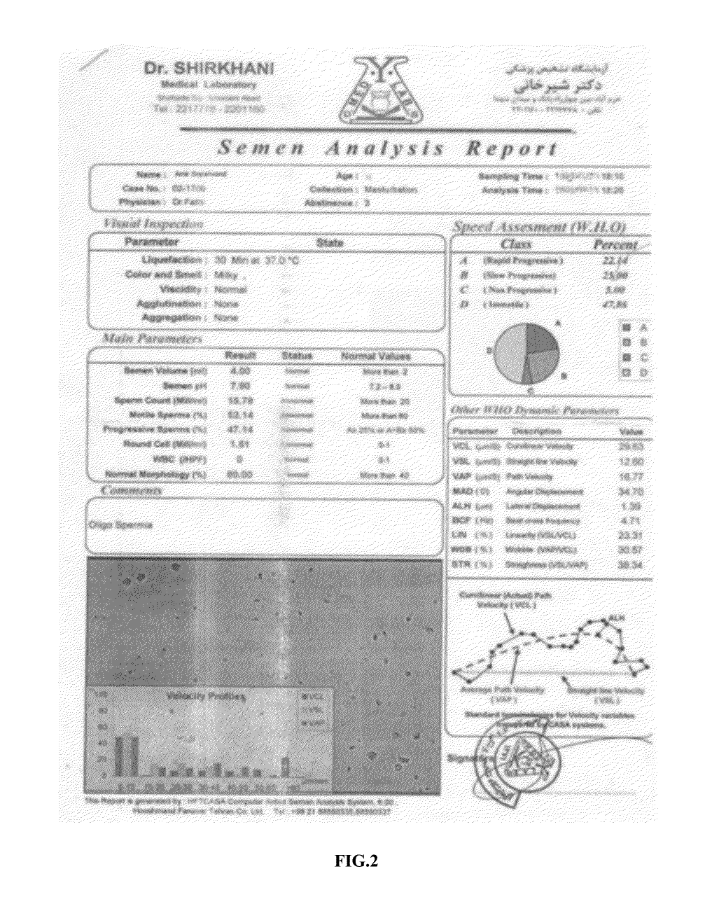

FIG. 2 illustrates a computer aided semen analysis system report showing semen analysis result of male patient after first month of administering herbal syrup, according to one embodiment herein. The FIG. 2 report illustrates the effect of herbal syrup (herbal syrup comprising Nigella sativa seed extract and Zingiber officinale root extract) administered for one month on sperm count, motile sperm and progressive sperms in the male patient with abnormal sperm parameters. The FIG. 2 report illustrates the sperm count in the semen of male patient is 15.78 million/ml of semen sample. The motile sperms in the male patient's semen sample are present in 52.14%. The progressive sperm in the male patient semen sample are present in 47.14%. The sperm count, motile sperm and progressive sperm are present below the normal levels or range recommended by WHO. The male patient has a condition of oligospermia.

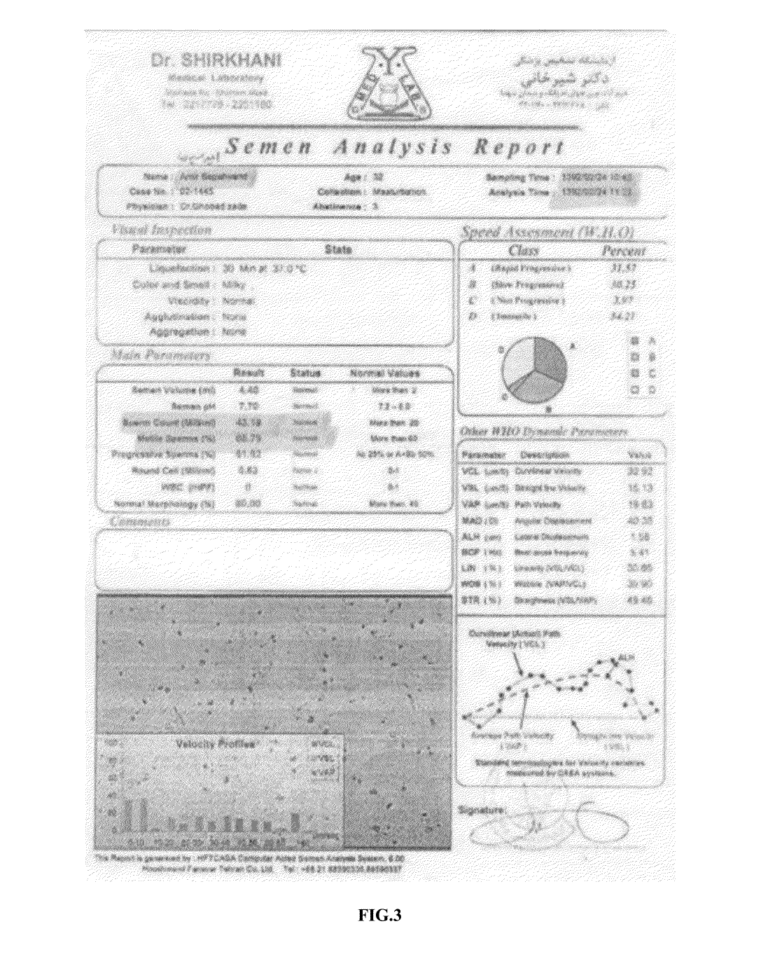

FIG. 3 illustrates a computer aided semen analysis system report showing semen analysis result of male patient after second month of administering herbal syrup, according to one embodiment herein. The FIG. 3 report illustrates the effect of herbal syrup (herbal syrup comprising Nigella sativa seed extract and Zingiber officinale root extract) administered for two month on sperm count, motile sperm and progressive sperms in the male patient with abnormal sperm parameters. The FIG. 3 report illustrates the sperm count in the semen of male patient is 43.19 million/ml of semen sample. The motile sperms in the male patients semen sample are present in 65.79%. The progressive sperm in the male patient semen sample are present in 61.62%. The sperm count, motile sperm and progressive sperm are present in a normal levels or range recommended by WHO. The male patient is not reported to have oligospermia.

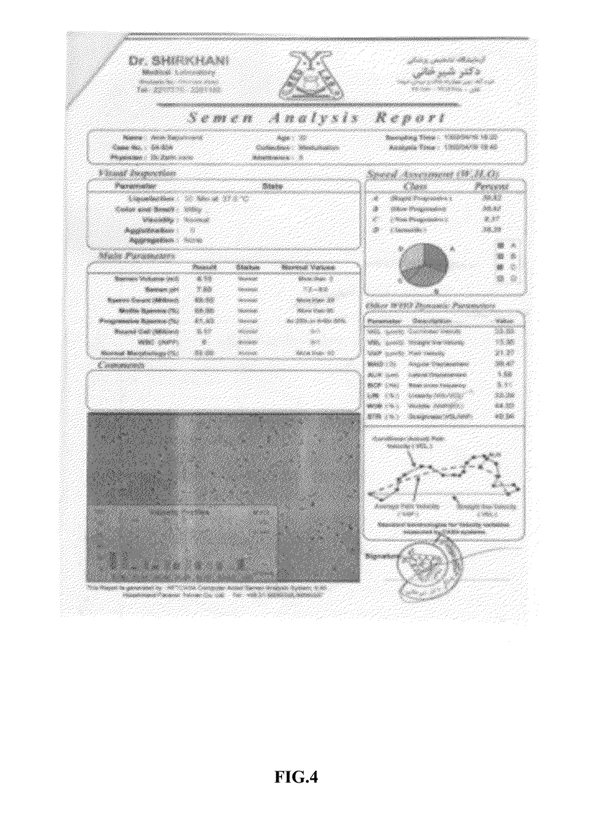

FIG. 4 illustrates a computer aided semen analysis system report showing semen analysis result of male patient after third month of administering herbal syrup, according to one embodiment herein. The FIG. 4 report illustrates the effect of herbal syrup (herbal syrup comprising Nigella sativa seed extract and Zingiber officinale root extract) administered for three month on sperm count, motile sperm and progressive sperms in the male patient with abnormal sperm parameters. The FIG. 4 report illustrates the sperm count in the semen of male patient is 89.52 million/ml of semen sample. The motile sperms in the male patients semen sample are present in 69.80%. The progressive sperm in the male patient semen sample are present in 61.43%. The sperm count, motile sperm and progressive sperm are present in a normal levels or range recommended by WHO. The male patient is not reported to have oligospermia.

The computer aided semen analysis system reports FIG. 2, FIG. 3 and FIG. 4 showing semen analysis result of male patient illustrate an increase in the sperm count, motile sperm and progressive sperms.

According to one embodiment herein, the ginger ethanol extract and the Nigella seed ethanol extract present in the herbal syrup composition has stereological effect on testis of the infertile male patients with abnormal sperm parameters. The active ingredient in the ginger ethanol extract is gingerol and the active ingredient in the Nigella seed ethanol extract is thymoquinone. The thymoquinone and gingerol has a positive effect on testis. The thymoquinone and gingerol stimulate testis of male patients to produce sperm cells and increase the motility of the sperm cells. The second group or control group of the male patients is administered placebo. The second group/control group male patients do not show any effect on the sperm production and sperm motility.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims submitted below. The scope of the invention will be ascertained by the following claims.

What is claimed is:

1. A method of synthesizing a herbal syrup composition, for targeting abnormal sperm parameters in infertile male patients, the method comprises the steps of:
collecting Nigella sativa seeds and Zingiber officinale roots;
cleaning and disinfecting the Nigella sativa seeds and the Zingiber officinale roots;
grinding the Nigella sativa seeds and Zingiber officinale roots separately to obtain Nigella sativa seed powder and Zingiber officinale root powder;
extracting thymoquinone from Nigella sativa seed powder using an alcohol solvent in a percolator machine, and wherein Nigella sativa seed powder alcohol extract is obtained in the percolator machine, and wherein the thymoquinone is an active ingredient of Nigella sativa seed powder alcohol extract;
extracting gingerol from Zingiber officinale root powder using an alcohol solvent in a percolator machine, and wherein Zingiber officinale root powder alcohol extract is obtained in the percolator machine, and wherein the gingerol is an active ingredient of Zingiber officinale root powder alcohol extract;
collecting the Nigella sativa seed powder alcohol extract and the Zingiber officinale powder alcohol extract separately;
mixing the Nigella sativa seed powder alcohol extract and the Zingiber officinale powder alcohol extract in a predetermined concentration to obtain a mixture of Nigella sativa seed powder alcohol extract and the Zingiber officinale powder alcohol extract;
adding a plurality of additives to the mixture of Nigella sativa seed powder alcohol extract and the Zingiber officinale powder alcohol extract to obtain the herbal syrup composition; and
wherein the herbal syrup composition is administered to infertile male patients with abnormal sperm parameters, and wherein the infertile male patients with abnormal sperm parameters are subjected to sperm analysis.

2. The method according to claim 1, wherein the alcohol solvent is ethanol.

3. The method according to claim 1, wherein the herbal syrup composition is administered to infertile male patients with abnormal sperm parameters in an amount of 7 ml.

4. The method according to claim 1, wherein the herbal syrup composition is administered to infertile male patients with abnormal sperm parameters during morning meal, and wherein the morning meal is breakfast.

5. The method according to claim 1, wherein the herbal syrup composition is administered to infertile male patients with abnormal sperm parameters for a period of 4 months.

6. The method according to claim 1, wherein the herbal syrup composition comprises the Nigella sativa seed powder alcohol extract and Zingiber officinale root powder alcohol extract in a concentration of 1-10% v/v.

7. The method according to claim 1, wherein the herbal syrup composition comprises the plurality of additives in a concentration of 150-200 ml.

8. The method according to claim 1, wherein the sperm analysis of the male patients after one month of administering the herbal syrup composition illustrates a sperm count in a semen sample of male patient in a concentration of 15.78 million/ml, and wherein motile sperms in the male patient semen sample are present in a concentration of 52.14%, and wherein progressive sperm in the male patient semen sample are present in a concentration of 47.14%.

9. The method according to claim 1, wherein the sperm analysis of the male patients after second month of administering the herbal syrup composition illustrates a sperm count in a semen sample of male patient in a concentration of 43.19 million/ml, and wherein motile sperms in the male patient semen sample are present in a concentration of 65.79%, and wherein progressive sperm in the male patient semen sample are present in a concentration of 61.62%.

10. The method according to claim 1, wherein the sperm analysis of the male patients after third month of administering the herbal syrup composition illustrates a sperm count in a semen sample of male patient in a concentration of 89.52 million/ml, and wherein motile sperms in the male patient semen sample are present in a concentration of 69.80%, and wherein progressive sperm in the male patient semen sample are present in a concentration of 61.43%.

11. A herbal syrup composition for targeting abnormal sperm parameters in infertile male patients, the herbal syrup composition comprises:
a Nigella sativa seed powder alcohol extract, and wherein thymoquinone is an active ingredient present in the Nigella sativa seed powder alcohol extract;
a Zingiber officinale root powder alcohol extract, and wherein gingerol is an active ingredient present in the Zingiber officinale root powder alcohol extract; and
a plurality of additives;
and wherein the herbal syrup composition is administered to infertile male patients with abnormal sperm parameters, and wherein the infertile male patients with abnormal sperm parameters are subjected to sperm analysis.

12. The herbal syrup composition according to claim 11, wherein the herbal syrup composition is administered to infertile male patients with abnormal sperm parameters in an amount of 7 ml.

13. The herbal syrup composition according to claim 11, wherein the herbal syrup composition is administered to infertile male patients with abnormal sperm parameters during morning meal, and wherein the morning meal is breakfast.

14. The herbal syrup composition according to claim 11, wherein the herbal syrup composition is administered to infertile male patients with abnormal sperm parameters for a period of 4 months.

15. The herbal syrup composition according to claim 11, wherein the herbal syrup composition comprises the Nigella sativa seed extract and Zingiber officinale root extract in a concentration of 1-10% v/v.

16. The herbal syrup composition according to claim 11, wherein the herbal syrup composition comprises the plurality of additives in a concentration of 150-200 ml.

17. The herbal syrup composition according to claim 11, wherein the sperm analysis of the male patients after one month of administering the herbal syrup composition illustrates a sperm count in a semen sample of male patient in a concentration of 15.78 million/ml, and wherein motile sperms in the male patient semen sample are present in a concentration of 52.14%, and wherein progressive sperm in the male patient semen sample are present in a concentration of 47.14%.

18. The herbal syrup composition according to claim 11, wherein the sperm analysis of the male patients after second month of administering the herbal syrup composition illustrates a sperm count in a semen sample of male patient in a concentration of 43.19 million/ml, and wherein motile sperms in the male patient semen sample are present in a concentration of 65.79%, and wherein progressive sperm in the male patient semen sample are present in a concentration of 61.62%.

19. The herbal syrup composition according to claim 11, wherein the sperm analysis of the male patients after third month of administering the herbal syrup composition illustrates a sperm count in a semen sample of male patient in a concentration of 89.52 million/ml, and wherein motile sperms in the male patient semen sample are present in a concentration of 69.80%, and wherein progressive sperm in the male patient semen sample are present in a concentration of 61.43%.
